(19) United States
(12) Patent Application Publication
(10) Pub. No.: US 2012/0085357 A1
(43) Pub. Date: Apr. 12, 2012

(54) DEVICE FOR IMPEDING A PERSON FROM ASSUMING A SUPINE POSITION

(76) Inventor: Robert Ek, Jarfalla (SE)

(21) Appl. No.: 13/318,512

(22) PCT Filed: May 3, 2010
(86) PCT No.: PCT/SE10/00117

§ 371 Date: Dec. 12, 2011

(30) Foreign Application Priority Data
May 6, 2009 (SE) 0900613-1

(51) Int. Cl. A61F 5/37

(52) U.S. Cl. 128/871

(57) ABSTRACT

A device for impeding a person from assuming a supine position in bed or other resting support includes a cushion support and movement restriction means fixable at the cushion support and a user. The cushion support designed for being placed on a resting support such as a bed. When user's head is resting on a cushion disposed on or in the cushion support, the user cannot turn from one lateral position to the other lateral position via a supine position but may turn so via an abdominal position. The cushion support includes a flexible cover and a stiffening means disposed in the cover. The stiffening means can be inserted into the cover and removable therefrom. The device includes also first connection means having an extension in the cover in the transverse direction thereof and an extension outside the cover at both sides of the user. The first connection means is coupled with the stiffening means for connection of the movement restriction means.

### FIELD OF THE INVENTION

The present invention relates to a device for impeding a person from assuming a supine position in bed or other resting support.

### BACKGROUND OF THE INVENTION

Snoring is generally accepted to be an important medical problem. During snoring the airways are temporarily at risk of becoming occluded, and breathing to stop (apnea). This results in insufficient oxygenation of the blood, which in turn results in tiredness in an awakened state even if the person in question gets sufficient sleep. Snoring is promoted by a recumbent position, since the soft palate is displaced in a rearward direction in the throat, and is occluding it. A method of reducing snoring and thus sleep apnea comprises hindering the person from assuming a recumbent position. Various devices for this purpose are known (WO 98/23188 A1; EP 0 765 647 A2; DE 29 618 276 U1; WO 01/54634 A1). While the known devices fulfil their purpose to a varying degree there is room for improvement in regard of ease of handling, cleaning, mechanical stability and functionality, as well as even in regard of their suitability to be carried by the user for a night's rest away from home.

### OBJECTS OF THE INVENTION

An object of the present invention is to disclose a device for impeding a person from assuming a supine position, which is simple to use, easy to carry when travelling, which at the same time is ligth and stable as well as cheap to produce.

Another object of the present invention is that the parts in contact with the user are easy to clean, that is, that the device for impeding a person from assuming a supine position is hygienically designed.

Still further objects of the invention will become apparent from the following summary of the invention, a number of preferred embodiments thereof, as well as the annexed patent claims.

### SUMMARY OF THE INVENTION

According to the present invention is disclosed a device for impeding a person from assuming a supine position, comprising a cushion support and movement restriction means fixable at the cushion support and the user.

The cushion support comprises a cover with an upper wall formed by an upper flexible material and a substantially planar lower wall formed by a lower flexible material. It is preferred for the upper wall material and/or the lower wall material to comprise a textile. It is preferred for the upper wall and the lower wall to be congruent in form and to be joined at their periphery. It is also within the ambit of the invention that the textile is reinforced with, for instance supported by, a flexibel polymer material, for instance polymer foam. Also conceivable are reinforcements of rubber foam or other resilient polymer foams of the kind used in single-piece bed mattresses and bed cushions or in mattress and bed cushions comprising several layers of same or different polymer foam materials. In a top view the cushion support is preferably of T-form or of square or rectangular form with optionally rounded corners. When placing the cushion support on a bed or a resting support at the head end thereof, the opposite edges of the cushion support intended to extend parallel with the long sides of the bed or the resting support, in the following called lateral edges, are preferably shorter than the edges of the cushion support extending substantially in a transverse of the bed. “Transverse” as used herein relates to the direction across the bed or the resting support. “Longitudinal” as used herein relates to the direction along the bed or resting support. The cushion support of the invention is intended to be placed between a cushion and the bed linen on the upper face of the bed at the head end of the bed. Alternatively the cushion support comprises a cushion element or means for attaching a cushion to the upper face of the cover to prevent the cushion from gliding off the cushion element. The cover comprises stiffening means disposed in the cover. The stiffening means are disposed in a transverse direction of the cushion support and are removable from and insertable into the cover. For this purpose the cover comprises an insertion opening extending in a transverse direction of the cushion support, the opening being preferably closable, for instance by a zip fastener. It is preferred for the insertion opening to be arranged in the lower wall of the cover. The stiffening means is of a length, which is preferably only insubstantially smaller that the width of the support at the portion of the support where the stiffening means is arranged, for instance shorter by 2% or maximally by 5%. The cover comprises means hindering the stiffening means from being displaced within the cover, for instance seams or pockets. The stiffening means is carried out in metal or polymer or a combination thereof, and is substantially stiff. According to a particularly preferred aspect of the invention the stiffening means consists of two or more, preferably three or four, elements that can be put together and taken apart, which facilitates transport of the device, for instance when travelling.

The stiffening means comprises a first flexible connection means for connecting the movement restriction means. It is preferred for the first flexible connection means to have the form of bands, such as textile bands, ropes, thin wires and similar, and comprises coupling means arranged at its first and second end sections. With this first coupling means second coupling means of the movement restriction means is releasingly connectable. The flexible first connection means extends along the stiffening means, and is preferably guided through eyes arranged at the ends of the stiffening means. The sections of the first connection means extending from the eyes are preferably conducted through openings in the cover and extend from the cover at both sides of the cover, the sections extending from the cover being called herein terminal sections. The eyes are preferably arranged at the lower face of the cover.

The movement restriction means comprises two bands or belts coupled at each other in form of an 8 so as to form two loops; alternatively the movement restriction means comprises a garment of corresponding function, such as a vest according to WO 01/54634 A1. In a mounted state the loops are placed around the upper arms of the user, where the upper arm joins the shoulder, so that the central portion shared by the loops or the crossing portion of the garment becomes disposed on the back of the user. At each loop and at a distance from the crossing portion is attached a second connection means, such as bands, belts or similar. The first connection means has two free ends whereas the second connection means have only one free end each. The free ends of the first connection means can be releaseably coupled with the free ends of the second connection means, preferably with coupling means arranged at the first and/or the second connection means. It is preferred for the coupling means to allow to couple the free ends of the first and second connection means in pairs so as to make the effective length of the portions of the first and second connection means disposed between the respective insertion opening and the respective loop coupling site to be adjustable.

The flexible second connection means can comprise means facilitating their coupling with the first connection means, for instance belt buckles, female and male part of press buttons arranged in a row along the band, Velcro® fasteners, and similar. The movement restriction means can also be provided with means such as slits, Velcro® fasteners, etc., which facilitate the fixation of the movement restriction means and allow the size of the loops to be adjusted for adapting the movement restriction means to the form of the user's body.

The invention thus discloses a device for impeding a person from assuming a supine position in bed or on another resting support, comprising a cushion support and movement restriction means fixable at the cushion support and a user, the cushion support designed for being placed on the resting support so as to make its transverse direction coincide with that of the resting support to, with the user's head resting on a cushion disposed on or in the cushion support, prevent the user from turning from one lateral position to the other lateral position via a supine position but allowing the user to turn so via an abdominal position, the cushion support comprising a flexible cover and a stiffening means disposed in the cover, the stiffening means being insertable into the cover and removable from the cover, further comprising a first connection means having an extension in the cover in the transverse direction thereof and an extension outside the cover at both sides of the user, the first connection means being coupled with the stiffening means for connection of the movement restriction means. It is preferred for the cover to comprise retaining means for keeping the stiffening means in its lateral position. It is preferred for the retaining means to comprise pockets and/or seams. It is furthermore preferred for the stiffening means to consist of two or more dismountable and mountable elements. The stiffening means has preferably about the same or an insubstantially shorter length as the width of the cover portion in which it is arranged. According to a preferred aspect of the invention coupling means are attached to or attachable to the portions of the connection means extending from the cover. According to another preferred aspect of the invention the cover comprises an upper and a lower wall, the walls being flexible, substantially congruent in form and fixed at each other at their periphery. According to a third preferred aspect of the invention a slit is arranged in the lower wall of the cover. The slit is preferably closable by closing means such as zip fasteners, buttons, press buttons, hooks and eyes, Velcro® fasteners, and similar. It is preferred for the first connection means to comprise bands, belts, ropes and similar. It is also preferred for the first connection means to extend through penetrating openings arranged in end portions of the stiffening means. According to a further preferred aspect of the invention the first connection means extends through openings in one of the cover walls, preferably the upper cover wall. It is preferred for the movement restriction means to comprise two loops in form of an 8, the size of the loops being preferably adjustable. It is preferred for the loops to be formed by textile bands, belts or similar coupled to each other, or to be comprised by a textile mini-garment such as a vest. According to a fourth preferred aspect of the invention one each of second connection means are fixed at each loop. It is preferred that the first and the second connection means can be coupled with each other in pairs by means of co-operating coupling means. It is preferred for the first and/or second connection means to comprise tensioning means allowing their total length to be adjusted.

According to a further preferred embodiment of the invention, the stiffening means comprises an oblong flat central element and two oblong flat identical terminal elements defining a plane in a mounted condition from which the terminal elements can be bent away out of plane in one direction but not the opposite direction. The stiffening means is designed to be mounted in the cover so that bending upwards is prevented but bending downwards (in the direction of the bed or resting support) is not prevented. This embodiment of the stiffening element of the invention can advantageously be put together and taken apart within the cover.

The invention will now be explained in more detail by reference to a number of preferred embodiments illustrated in a drawing.

### SHORT DESCRIPTION OF THE FIGURES

It is shown in

FIG. 1 a first preferred embodiment of the stiffening means according to the invention, in a top view;

FIG. 1a the stiffening means according to FIG. 1 and in the same view, in a dismounted condition;

FIG. 1b a section A-A through the stiffening means according to FIG. 1, enlarged;

FIG. 2 stiffening means according to FIG. 1 and in the same view, with first connection means mounted;

FIG. 3 coupling means mounted at first and second connection means according to the embodiment of FIG. 1, in a enlarged sectional view B-B;

FIG. 4 a first embodiment of the cushion support according to the invention, in a top view, a portion of the lower cover wall and of the stiffening insert inside this wall removed;

FIG. 5 the embodiment of FIG. 4, in a bottom view;

FIG. 6 a second embodiment of the cushion support according to the invention, in a bottom view;

FIG. 7 a first embodiment of the movement restriction means according to the invention resting on a flat support, in a schematic presentation and in top view;

FIG. 7a the movement restriction means according to FIG. 7, in a real presentation, in a perspective view;

FIGS. 8, 8a the movement restriction means according to FIG. 7 coupled to the cushion support according to FIG. 7 and mounted on a user, in a first (8) and a second (8a) perspective view;

FIG. 9 a second embodiment of the movement restriction means according to the invention, in a real presentation and in a perspective view;

FIGS. 10, 10a the movement restriction means according to FIG. 8, mounted on a standing user, in a front view and a rear view;

FIGS. 11-11c a second embodiment of the stiffening means according to the invention, in a top view (FIGS. 11, 11a), a side view (11b) and in section (11c);

FIG. 12 a third embodiment of the cushion support according to the invention, in a top view, a central portion of the upper wall of the cover removed and with an inflatable cushion disposed in the cover.

FIG. 13 the central element of a further embodiment of the stiffening means according to the invention, in a perspective view from above;

FIG. 14 a terminal element of the embodiment of the stiffening means of FIG. 13.

### DESCRIPTION OF PREFERRED EMBODIMENTS

### Example 1

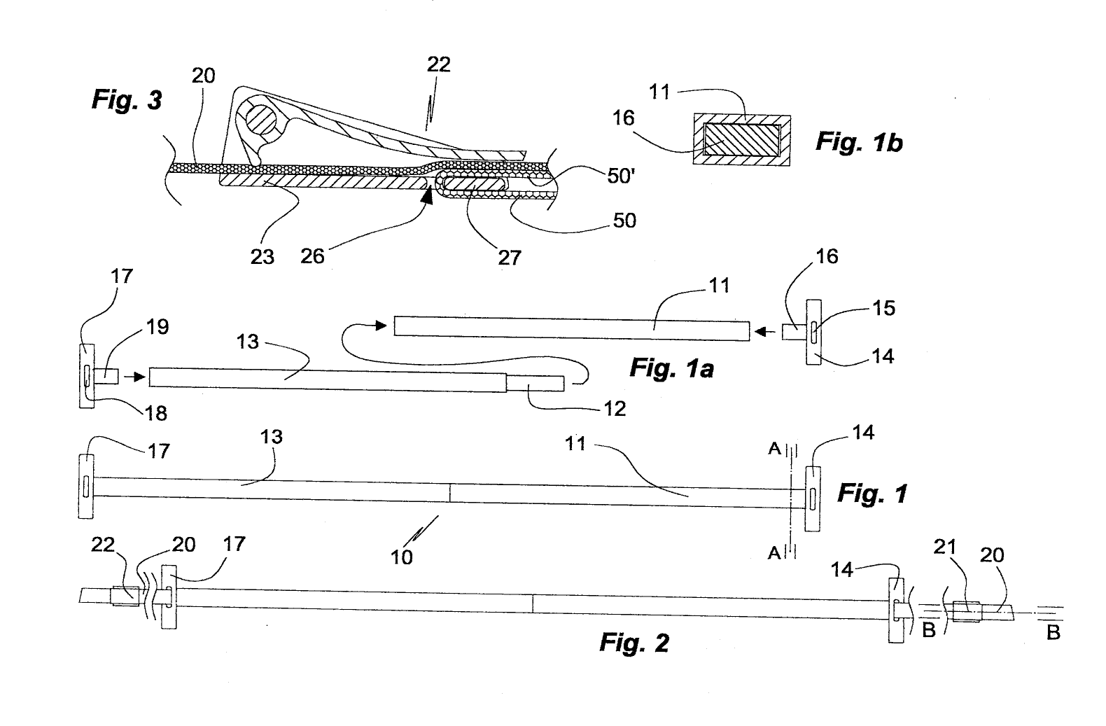

The preferred embodiment of the stiffening means 10 according to the invention shown in FIGS. 1, 1a consists of four parts: a right 14, 15, 16 and a left 17, 18, 19 guide element, and a right 11 and a left 12, 13 rib. The arrows in FIG. 1a indicate the assembly of the guide elements and ribs. The ribs consist of a transverse beam 14, 17 with a through bore (eye) 15, 18, each having a pin 16; 19 insertable into the rectangular-profile lumen of the ribs 11; 13, the sectional lumen area of which is slightly larger than that of the dowels 16; 19, cf FIG. 1b, section A-A (FIG. 1). The ribs 11; 13 are assembled in a similar manner by means of pin 12, which is inserted into the lumen of rib 11.

In FIG. 2 are shown connection means 20, 21, 22 according to the invention mounted on stiffening means 10. The connection means consist of a woven stretching band or strap 20 extending along the bottom side of the ribs 11, 13 and then through eyes 15, 18. At both ends of the band stretching band locks 21, 22 are mounted, which locks can be opened and locked, and thus are displaceable along terminal portions of the band 20 (FIG. 3). The stretching band lock 21 is shown in a locked state.

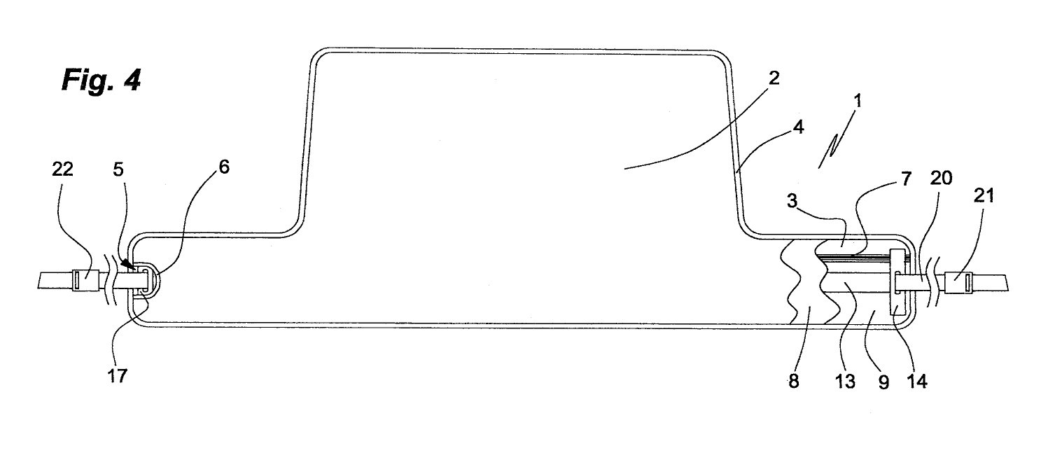

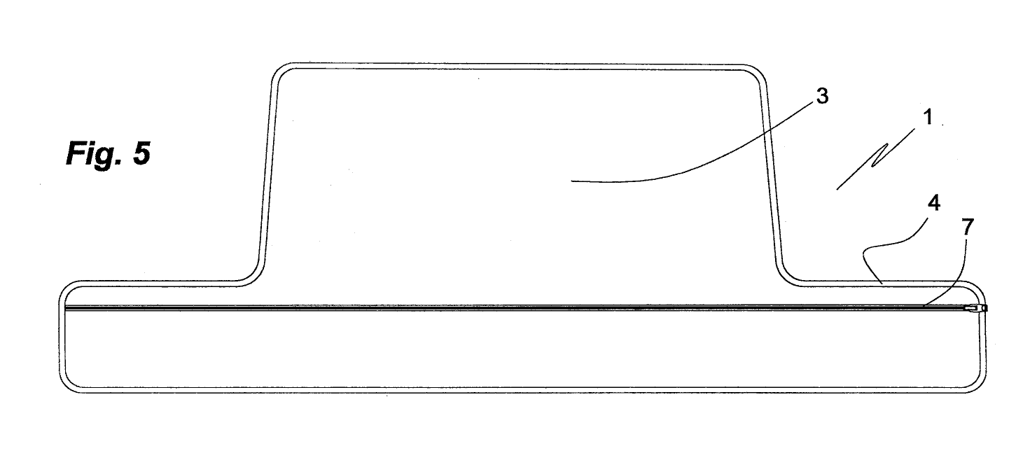

FIGS. 4 and 5 show a first preferred embodiment of the cushion support 1 according to the invention with stiffening means 10 and the first connection means mounted in a cover 2, 3 with an upper wall 2 and a lower wall 3 in a woven textile. The walls 2, 3 of the cover are congruent in form and connected by sewing along their periphery profided with a folded-over edge strip. 4. The cover has the form of a T with a short and thick central beam. The cover 2, 3 is armed at its inside with flexible foam material inserts 8, 9, which abut against the inner face of the upper wall 2 and against the inner face of the lower wall 3, respectively.

At the transverse beam portion the lower wall 3 has a transverse opening provided with a zip fastener 7, the opening extending over the entire width of the transverse beam portion. While the insert 8 is congruent in form with the upper wall 2, the insert 9 is limited to the portion of the transverse beam disposed between the zip fastener 7 and the edge of the transverse T-beam extending in a transverse direction. The end portions of the strap 20 with their strap locks 21, 22 extend from the openings 17, 18, respectively, in the upper wall at the transverse ends of the transverse T-beam. Through the opened zip fastener 7 the stiffening means 10 with the strap 20 mounted in the eyes 15, 18 can be inserted easily with their terminal portions 14, 15; 17, 18 into the pockets formed by the terminal portions of the transverse T-beam, and be kept there by closing the zip fastener 7. In connection with the insertion of the stiffening means 10 the ends of the strap 20 are guided through the openings 5, 5′ in the upper wall 2 and the corresponding insert 9. Thereafter the snap locks 21, 21 are mounted on the respective end portions of the strap 20 disposed outside of the cushion support. When travelling the user might, for instance, desire to transport the cushion support in a small suitcase or similar; the stiffening means then can easily be disassembled and put back in portions into the pocket; for doing this it is not necessary to dismount the snap locks 21, 22. The edges of openings 5, 5ä′ are reinforced by textile bands folded over and fixed by sewing.

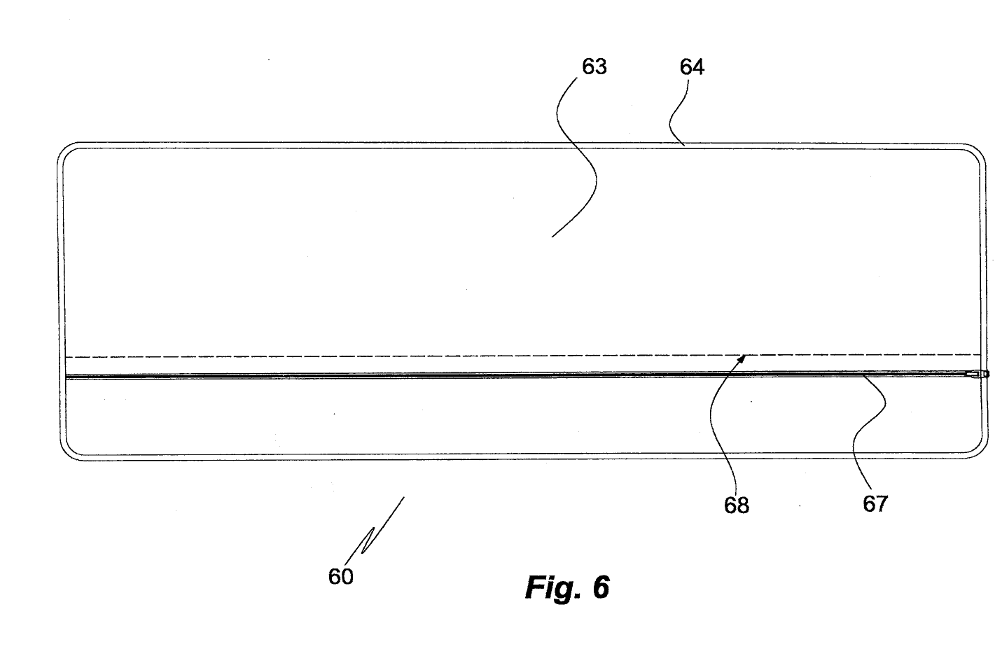

A second preferred embodiment of the cushion support 60 according to the invention is shown in FIG. 6. The cushion support 60 is rectangular in form with rounded corners; its walls are connected by sewing along their periphery at a folded-over edge strip 64. A zip fastener 67 is mounted at an opening in the lower wall 63 of the support, the opening extending in a transverse direction over the entire width of the cushion support 60. A seam 68 divides the inner volume of the cushion support 60 into two voids, of which the one with the zip fastener 67 forms the pocket for disposing the stiffening means (not shown), which can be of same kind as those of the first embodiment. The seam 68 limits the mobility of the stiffening means in the cushion support 60.

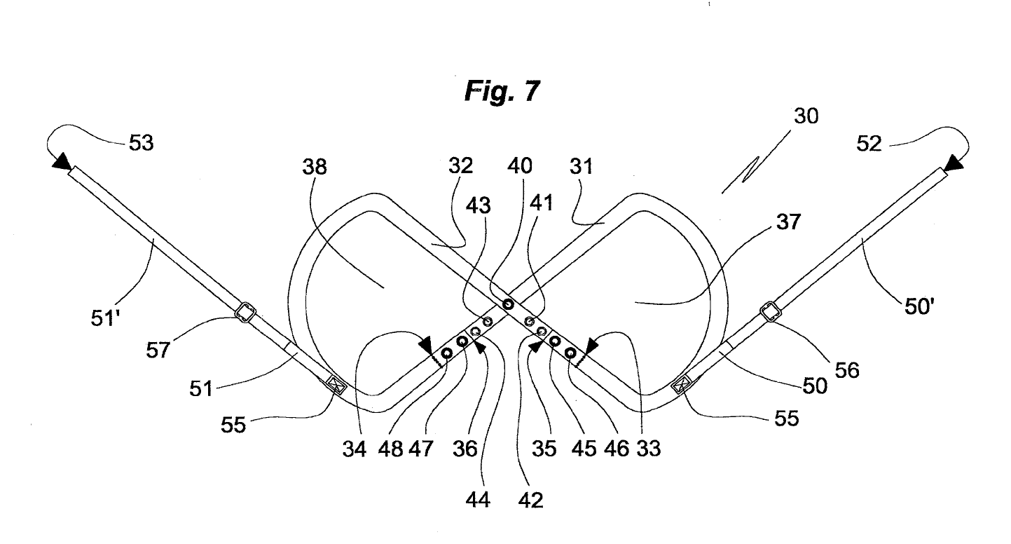

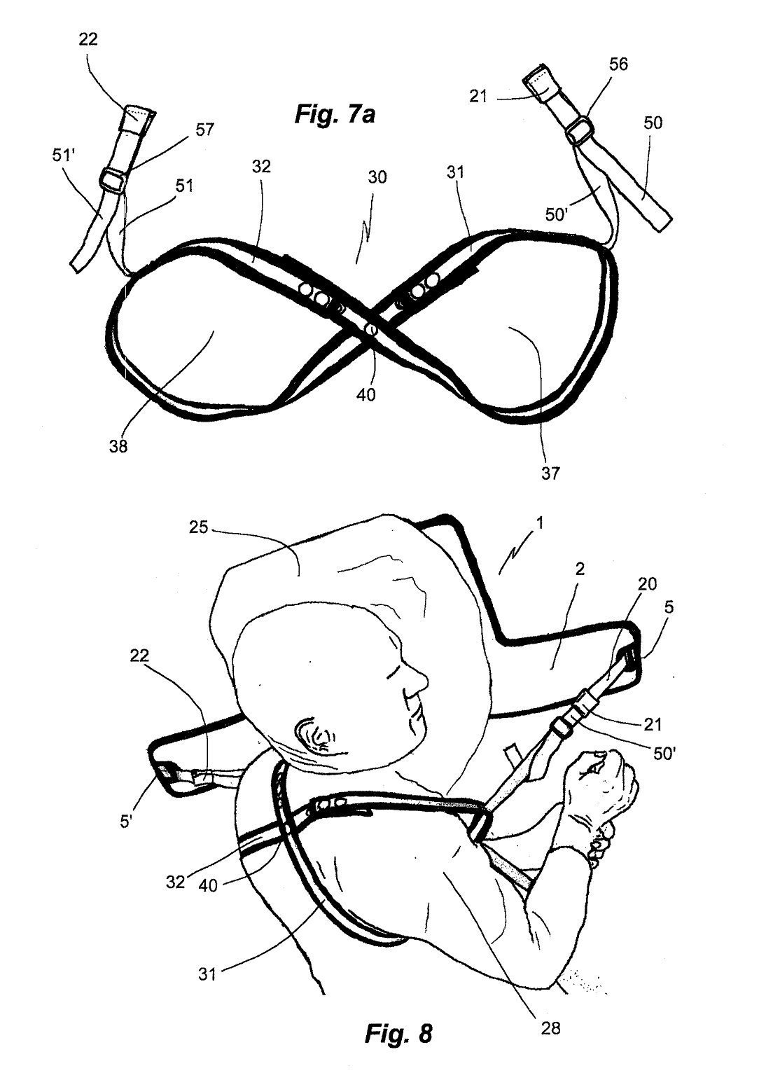

In FIG. 7 is shown a rough sketch of a first embodiment of the movement restriction means 30 according to the invention. The movement restriction means 30 comprise two woven eye bands 31, 32, which are releaseably coupled with each other under formation of a circle, coupling being achieved by means of press buttons arranged in rows on and along the eye bands 31, 32. By locking a pair of diametrically disposed male and female press buttons 40 the circle is transformed into the ∞ shown in FIG. 7.

The first eye band 31 extends from the terminal edge 35, which can be seen in FIG. 7, to the terminal edge 34 obscured by the other eye band 32. At the other eye band 32 the visible terminal edge has reference no. 36, and the obscured terminal edge reference no. 33. The number of press buttons is substantially higher than required for coupling. The purpose with the higher number of press buttons is to allow the size of eyes 37, 38 to be varied so as to adapt it to the user's bodily constitution. At the visible end of the first eye band 31 two male press buttons 35, 45 are arranged, which can be coupled with female press buttons 41, 42 and two obscured female press buttons at the obscured end of the other eve band. In a corresponding manner are arranged two male press buttons 37, 38 at the obscured end of the other eye band 32, which can be coupled with female press buttons 43, 44 and two obscured female press buttons at the obscured end of the first eye band 31. Thus each eye band 31, 32 extends over somewhat more than a hemicircle, the terminal portions extending from the hemicircle overlap with corresponding terminal portions of the other eye band in a manner that, when positioning the movement restriction means on a flat support, one of the two ends of each eye band 31, 32 is overlapped by one end of the other band and thus obscured in the figure. At each of the eye bands 31, 32 one woven strap 40, 50′; 51, 51′ is secured (by sewn seams 55). Each of the straps 50, 50′; 51, 51 forms a loop by means of friction buckles 56, 57. Loop sections 50, 51 disposed adjacent to the eye bands are partially obscured in FIG. 7 by end portions 50′, 51′ of the loops. The loops extend through transverse slits 26 in the bottom plates of the strap locks 21, 22 so as to form circles and affect transverse ribs 27 of the respective bottom plate 23 (FIG. 3). The size of the loops and thus the effective length of the straps 50, 50′; 51, 51′ from the seam fixations 55 to the loop ends 52, 53 can be adjusted by means of tensioning buckles 56, 57. FIG. 7a illustrates the movement restriction means, in a real presentation and with their loops 50, 50′; 51, 51′ mounted at the strap locks 20; 21.

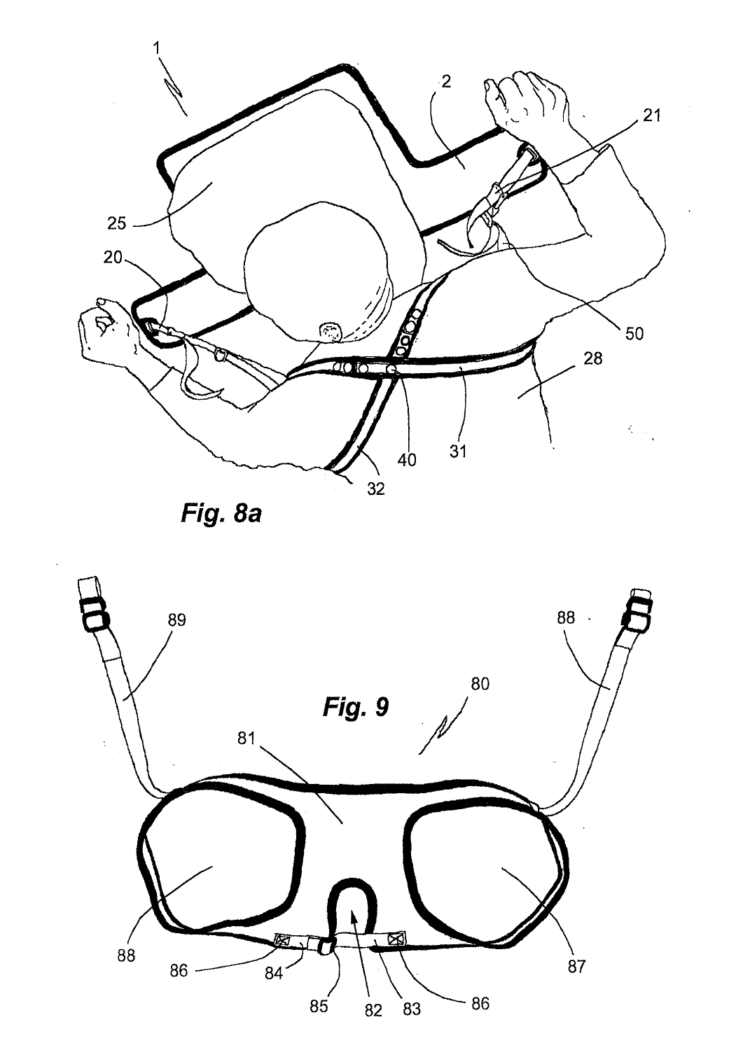

FIGS. 8 and 8a show a user 100 with mounted movement restriction means 30 resting with his head on a cushion 25 in left and right lateral positions, respectively. Via the snap locks 21, 22 the movement restriction means 30 are coupled with the first connection means strap 20 extending from cushion support 10 disposed under the cushion 25. The length of the straps 50′, 51 of the movement restriction means are adjusted so as to prevent the user 28 from assuming a supine position and turning, via a supine position, to the other side to assume the opposite lateral position. The user is however not prevented from turning to the other side via an abdominal position.

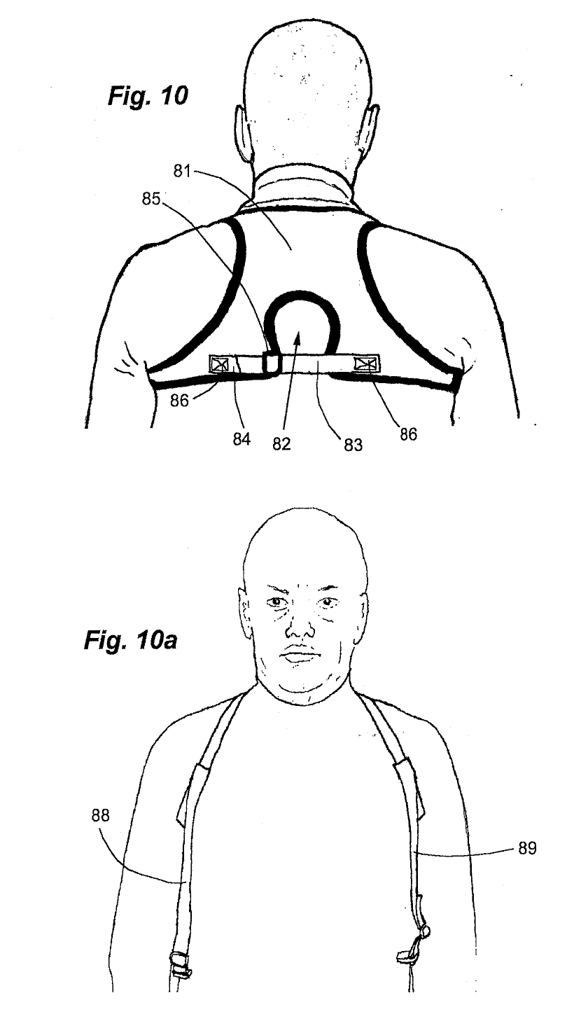

In FIG. 9 is shown a second embodiment of the movement restriction means according to the invention. The movement restriction means 80 comprises a mini-vest with openings 87, 88 for the arms. A central slit 82 is arranged on the back portion of the mini-vest, the width of which can be adjusted to the physique of the user by belts 83, 84 attached by seams 86 disposed at opposite sides of the slit 82, one belt 83 comprising a friction buckle 85 into which the other belt 84 is inserted and by means of which its length is adjustable. The straps 88, 89 fastened at the front portion of the garment have the same function as the straps of the embodiment according to FIGS. 7-8a. FIG. 10, 10a illustrates a standing user with the vest 81 mounted and the width of the slit 82 adjusted to fit the user's physique.

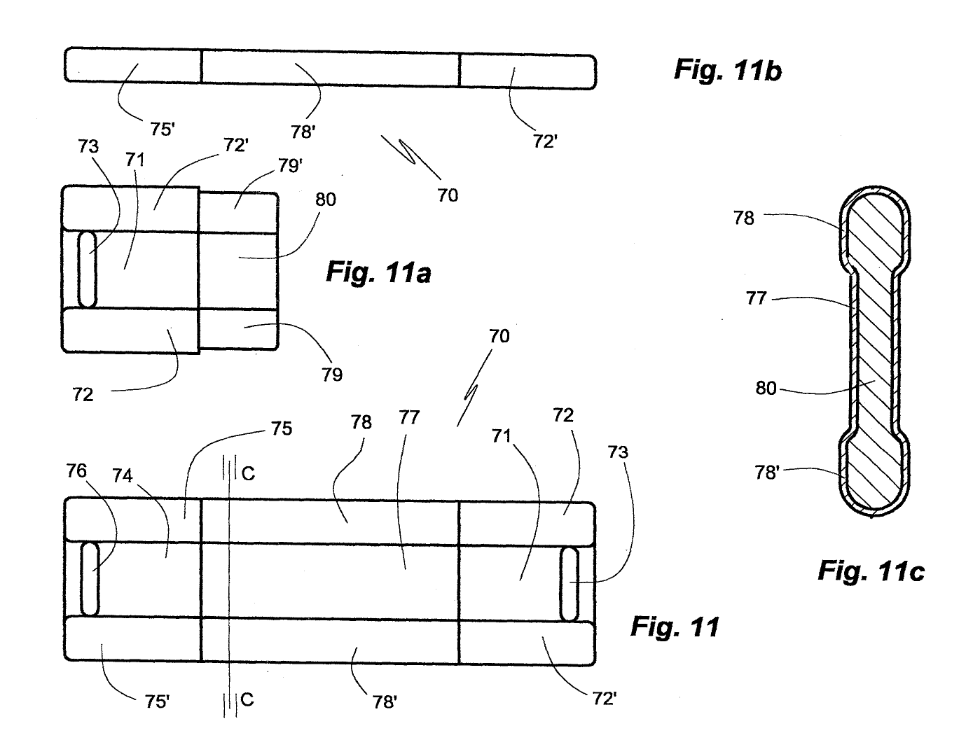

A second embodiment of the stiffening means according to the invention is shown in FIGS. 11-11c. The stiffening means 70 can be carried out in a metal, such as steel plate, or a hard polymer, such as polystyrene. The stiffening 70 means is built up in three elements, which can be put together and taken apart: a hollow central element 77, 78, 78′ and two identical terminal elements 71, 72, 72′; 74, 75, 75′. In a top/bottom view (FIG. 11) and a side view (FIG. 11b) the stiffening means appears as substantially rectangular and have the same profile in section (FIG. 11c) over their entire length. The stiffening means 70 is relatively flat. A central portion 17; 77; 74 of minor thickness is surrounded on both sides by elevations 72, 78, 75; 72′, 78′, 75. The valley thus formed has a suitable width for guiding a strap (not shown) corresponding to the strap 20 in the embodiment according to FIGS. 1-3. Eyes 73; 76 are arranged in the terminal elements 71, 72, 72′; 74, 75, 75′ for leading through the strap. As illustrated in regard of the terminal element 71, 72, 72′ the terminal elements 71, 72, 72′; 74, 75, 75′ are mountable in the central element 77, 78, 78′ by means of dowels 79, 79′, 80 insertable into the lumen of the central element.

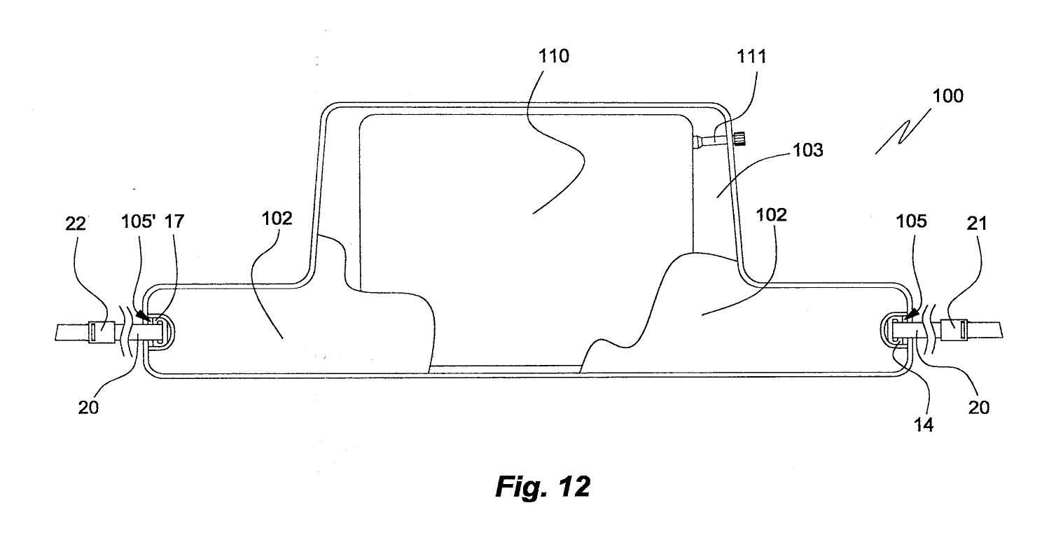

FIG. 12 illustrates a third preferred embodiment 100 of the cushion support according to the invention provided with an inflatable cushion 110 comprising a valve 111. Otherwise the cushion support 100 is similar to the cushion support 111, except for the stiffening inlays 8 and 9 of the first embodiment lacking and the support 100 comprising, in its rear wall 103, a slit (not shown) instead of the zip fastener 1, the slit as overlapping edges and extends over the entire width of the cushion support 100. The slit can be closed by buttons and corresponding buttonholes, hooks and eyes or other suitable closing means. The absence of stiffening inserts and zip fastener facilitates cleaning of the cushion support 100. The already described stiffening means 10 is mounted in the cushion support, its terminal elements 14, 17 being partially visible in the openings 105, 105′ in the front wall 102 through which the strap 20 extends. At both of its ends the strap 20 is provided with snap locks 21, 22.

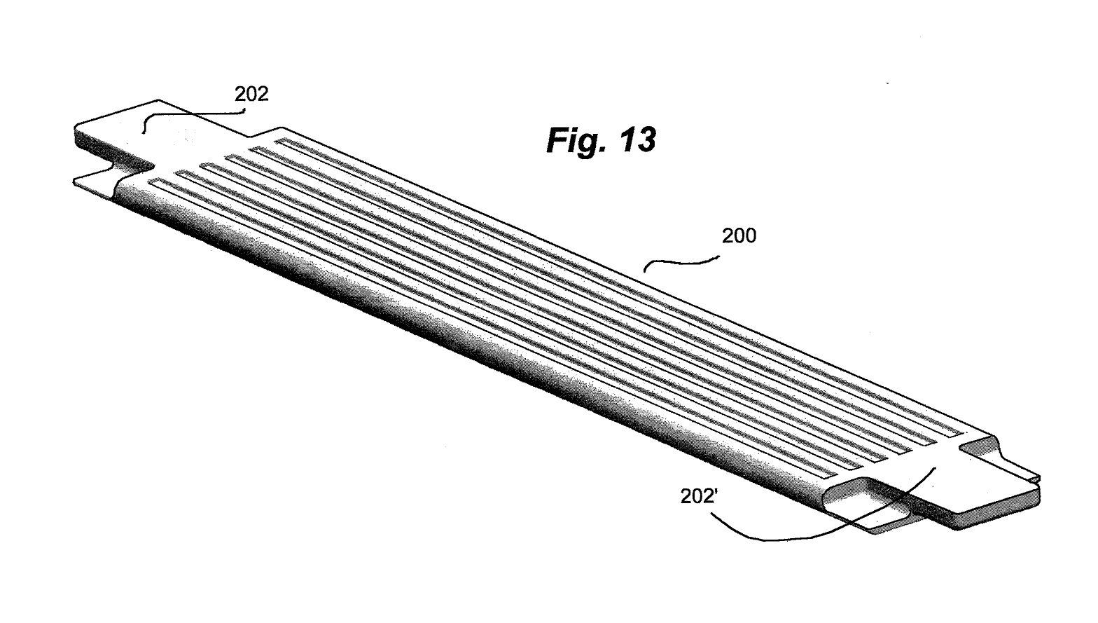

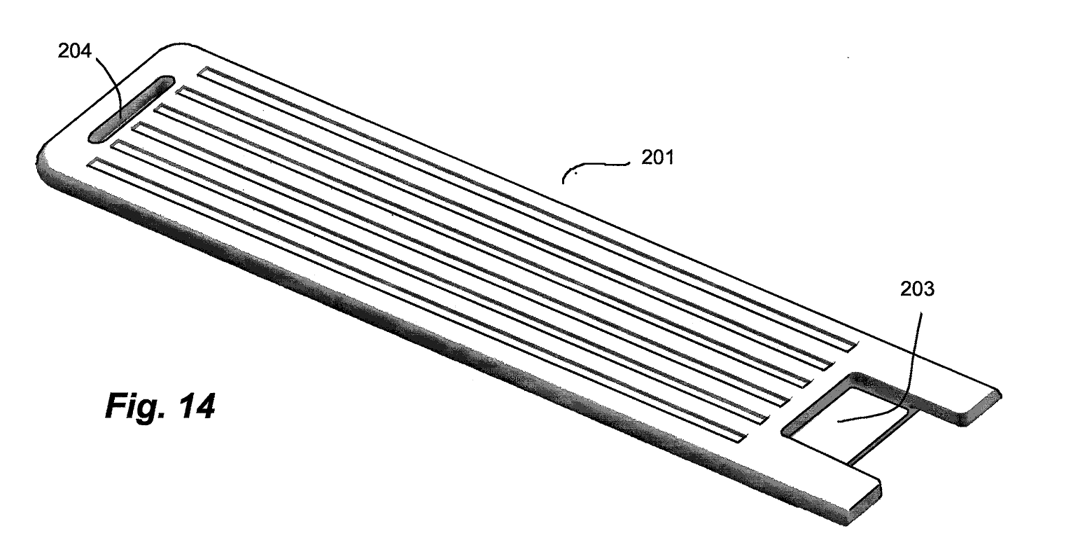

FIGS. 13 and 14 illustrate a further embodiment of the stiffening means according to the invention, comprising an oblong flat central element 200 comprising identical connectors 202, 202′ at its ends and two identical oblong flat terminal elements 201 each comprising a connector 203 at one end and a through opening 204a the other. The elements 200, 201 are of a stiff polymer material such as polycarbonate or polystyrene. They may be reinforced by metal, in particular steel, inlays. The terminal elements 201 are mounted on the central element 200 by inserting the connectors 202, 202′ of the central element 200 into their connectors 203. The connectors 202, 203 and 202′, 203 co-operate in a manner allowing the terminal elements to be bent away from the viewer while preventing them to be bent towards the viewer. This facilitates the dismounting of the stiffening means inside of the cover, that is, the stiffening element does not have to be removed from the cover for dismounting and assembling.

1. Device for impeding a person from assuming a supine position in bed or on another resting support, comprising a cushion support and movement restriction means fixable at the cushion support and a user, the cushion support designed for being placed on the resting support so as to make its transverse direction coincide with that of the resting support to, with the user's head resting on a cushion disposed on or in the cushion support, prevent the user from turning from one lateral position to the other lateral position via a supine position but allowing the user to turn so via an abdominal position, the cushion support comprising a flexible cover and a stiffening means disposed in the cover, the stiffening means being insertable into the cover and removable from the cover, further comprising a first connection means having an extension in the cover in the transverse direction thereof and an extension outside the cover at both sides of the user, the first connection means being coupled with the stiffening means for connecting of the movement restriction means.

2. The device of claim 1, wherein the cover comprises a means for keeping the stiffening means in its transverse position.

3. The device of claim 1, wherein the position keeping means comprise pockets and/or seams.

4. The device of claim 1, wherein the stiffening means consist of two or more elements that can be put together and taken apart.

5. The device of claim 1, wherein the stiffening means has about the same length as the width of the portion of the cover in which it is arranged, or a slightly shorter length.

6. The device of claim 1, wherein the coupling means is fixed or fixable at sections of the connection means extending from the cover.

7. The device of claim 1, wherein the cover comprises an upper wall and a lower wall, the walls being flexible, substantially congruent in form, and fixed at each other along their periphery.

8. The device of claim 7, comprising a slit in the lower wall of the cover.

9. The device of claim 8, wherein the slit is closable by closing means disposed at the edges of the slit, such as zip fastener, buttons, press buttons, hooks and eyes, Velcro® fasteners.

10. The device of claim 1, wherein the first connection means comprises bands, belts, cords, and the like.

11. The device of claim 1, wherein first connection means extends through penetrating openings arranged in terminal portions of the movement restriction means.

12. The device of claim 1, wherein the first connection means extends through openings in one of the cover walls, preferably in the upper cover wall.

13. The device according to claim 1, wherein the movement restriction means comprises two loops in form of an eight.

14. The device according to claim 13, wherein the size of the loops is adjustable.

15. The device according to claim 13, wherein the loops are formed by textile bands, belts or similar coupled with each other or are comprised by a textile mini-garment such as a vest.

16. The device according to claims 13, wherein second connection means are fixed at the loops.

17. The device according to claim 16, wherein the first and second connection means can be coupled in pairs by co-operating coupling means.

18. The device according to claim 17, wherein the first and/or second connection means comprise tensioning means allowing adjustment of their total length.

19. The device of claim 4, wherein the stiffening means comprises at least two elements joined to each other by a dismountable connection allowing them, when disposed in a planar mounted state to be bent out of plane in one direction but not in the opposite direction.
